(19) United States
(12) Patent Application Publication
(10) Pub. No.: US 2026/0233027 A1
(43) Pub. Date: Aug. 13, 2026

(54) WIDE FIELD OPHTHALMIC DEVICES FOR LIGHT THERAPY

(71) Applicant: Lumos Health Inc., Waterloo (CA)

(72) Inventors: Jamie Marc ZEITZER, Stanford, CA (US); Lucas Wen TANG, Bellevue, WA (US); Zhenle CAO, Cambridge, MA (US); Hyunje RO, Ontario (CA)

(21) Appl. No.: 19/647,676

(22) Filed: Apr. 14, 2026

Related U.S. Application Data
(63) Continuation of application No. PCT/US2024/050848, filed on Oct. 10, 2024.
(60) Provisional application No. 63/592,214, filed on Oct. 23, 2023.

(51) Int. Cl. A61N 5/06; G02C 11/00

(52) U.S. Cl. CPC A61N 5/0622; G02C 11/10; A61N 2005/063; A61N 2005/0651; A61N 2005/0665

(57) ABSTRACT

Methods, systems, and devices related to light therapy using ophthalmic lenses are disclosed. In one example aspect, an optical device includes at least one light source, and a system of light guiding optics configured to direct a light from the at least one light source to a back of a human retina for different therapeutic effects. The light entering the human retina is configured to span a field of view that is approximately up to 220 degrees horizontally and 135 degrees vertically, and the light entering the human retina from the at least one light source is controlled in a manner to avoid a central area that is around +/−10 degrees from a center of the human retina.

### CROSS-REFERENCE

This application is a continuation of International Application No. PCT/US2024/050848, filed Oct. 10, 2024, and entitled “WIDE FIELD OPHTHALMIC DEVICES FOR LIGHT THERAPY,” which claims the benefit of priority to U.S. Provisional Application No. 63/592,214, filed Oct. 23, 2023, and entitled “WIDE FIELD OPHTHALMIC DEVICES FOR LIGHT THERAPY,” which are assigned to the assignee hereof. The disclosure of the prior Applications are considered part of and are incorporated by reference in this Patent Application.

### TECHNICAL FIELD

This patent document relates to the field of wearable ophthalmic devices for performing different types of light therapies.

### BACKGROUND

Ophthalmic devices (e.g., wearable glasses, contact lenses) are used to help correct visual refractive errors and help people see more clearly. Using materials with different refractive indices, these lenses bend light to help the eye properly focus images.

Advancements in technology bring about additional uses for ophthalmic devices, such as augmented reality, where films and coatings added to the lenses combined with artificial light help superposition images and videos over normal vision. Another application that expands of the ophthalmic lenses is the field of light therapy, where electronic light sources work together with the lenses to provide additional light to the user to provide treatment.

### SUMMARY

This patent document describes, among other things, techniques that relate to ophthalmic devices that can provide therapeutic light to selective areas of the retina intended for photosensitive ganglion cell activation. In addition, methods to ensure the device's discreteness and privacy to the user from an external observer.

In one example aspect, an optical device includes at least one light source, and a system of light guiding optics configured to direct a light from the at least one light source to a back of a human retina for different therapeutic effects. The light entering the human retina is configured to span a field of view that is approximately up to 220 degrees horizontally and 135 degrees vertically, and the light entering the human retina from the at least one light source is controlled in a manner to avoid a central area that is around +/−10 degrees from a center of the human retina.

In another example aspect, an ophthalmic device with light source(s) which has the capability to project across the entire surface of the inner surface of the lens of the glasses, with the ability to selectively turn off its projection in certain areas. A custom reflector system at the inner lens surface is used to redirect the source light onto the retina across a wide field of view.

In another example aspect, some light sources project light directly into the user's retina from an oblique angle, this light can be mounted and hidden in the frame of the glasses, or embedded in the lenses of the glasses, while other light sources can project light onto a system of mirror(s), which redirect the light into the user's eyes.

In another example aspect, light can travel through the lenses of the glasses using total internal reflection, and coupled out of the lenses to reach the user's eyes. Unlike traditional waveguides, the coupling can be done in such a manner as to not preserve the imaging information of the original light source, rather, that the coupling is done in a way to ensure that light can reach a specifically targeted location of the retina for therapeutic purposes.

In another example aspect, an eye tracking system can be used, as a standalone, or in conjunction with the optical projection system, where the eye tracking can may be used as input controls to the light and a reflector system such that it can adapt the retinal projection of light based on eye movements. Examples of reflector system methods include MEMS micromirror, LCOS, mechanical mirror, tunable mirrors (liquid crystal), and/or other types of spatial light modulators.

In another example aspect, the methods described in this patent document can be used in tandem with other augmented ophthalmic glasses technologies such as waveguides, direct light projection, phase arrays, mirror-based laser scanning, electro-optic modulators, risley prisms, and/or other reflection based light therapy methods, to create a holistic solution for light therapy.

These, and other, aspects are described in the present document.

### BRIEF DESCRIPTION OF THE DRAWINGS

FIG. 1A illustrates an example optical device in accordance with one or more embodiments of the present technology, with a reflector system mounted on the inner surface of the spectacle lens coupled with a light source.

FIG. 1B illustrates a ray trace diagram in accordance with one or more embodiments of the present technology, showing reflector angles for proper retinal projection.

FIG. 2A illustrates an example optical system in accordance with one or more embodiments of the present technology.

FIG. 2B illustrates another example optical system in accordance with one or more embodiments of the present technology.

FIG. 3 illustrates an example optical system in accordance with one or more embodiments of the present technology, where light sources work together with a system of reflectors to deliver light at oblique angles to wide field of view locations on the retina.

FIG. 4A illustrates a plot showing an example angular distribution of photosensitive cells on the human retina.

FIG. 4B illustrates a plot showing an example distribution density of ipRGCs on the human retina.

FIG. 4C illustrates an example reference on how the angles shown in FIG. 4A and FIG. 4B match to a cross section of a human eyeball.

FIG. 5 illustrates an example optical system in accordance with one or more embodiments of the present technology, where the light sources and reflection systems work in tandem with sensors on the device to enable eye tracking.

FIG. 6 is an example optical system that includes a frame accordance with one or more embodiments of the present technology.

FIG. 7 is a block diagram that illustrates an example of a computing system in which at least some operations described herein can be implemented.

### DETAILED DESCRIPTION

Wavelength based reflection and transmission can have special uses in light therapy. For example, different wavelengths of light can have different therapeutical effects on users. This patent document discloses techniques that can be implemented in various embodiments to provide an optical device to provide different types of light therapies (e.g., to help maintain the right sleep-wave cycle, regulate emotions, and/or boost performance) without introducing any disturbance to the user's daily activities.

Human vision begins with the capture of photons by the retina, a fine layer of cells in the back of the eyes. Cone cells within the eyes are responsible for high accuracy central color vision. There are three types of cone cells that are responsive for different parts of the visible spectrum of light, also called wavelengths. In addition to the cones, which are responsible for vision of color, the eyes have rod cells that provide low light and peripheral vision. When a photon is captured by these photoreceptor cells, it triggers a complex series of reactions, turning light in the external world into a neural signal inside the brains.

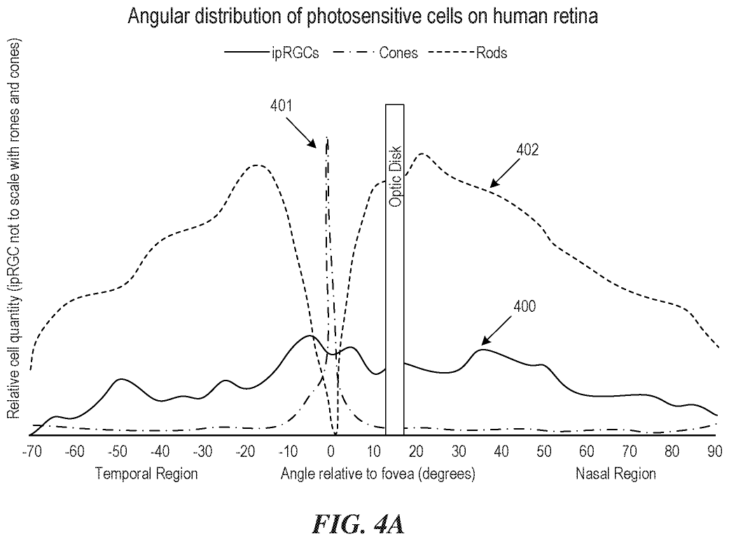

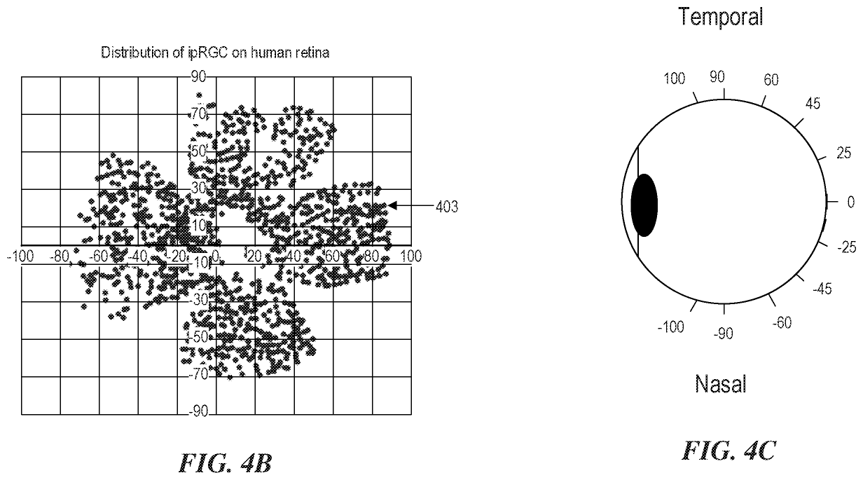

In addition to cones and rod cells, an important cell responsible for much of the effects of light therapy is the intrinsically photosensitive retinal ganglion cell (ipRGC), which has been shown to have a significant impact on human circadian rhythm, mood, and sleep. FIG. 4A illustrates an example distribution of ipRGCs compared with cones and rod cells across the temporal nasal (horizontal) axis of the eye. FIG. 4B shows an example distribution of ipRGCs on the flattened human retina. FIG. 4C provides an example reference of how the angles on FIG. 4A and FIG. 4B match to a cross section of a human eyeball. Given that the cones responsible for color and working vision is generally around the +/−10 degree of the human retina (fovea region), it is generally a good idea for a pair of light therapy glasses to be able to limit the amount of light entering that region. The rest of the retina, spanning all 200 degrees of the human horizontal field of view, provides great locations for therapeutic light, given the presence of abundant ipRGC across a wide span as shown in FIG. 4A and FIG. 4B.

Conventional augmented reality waveguides used in smart glasses are able to provide up to +/−25 degree field of view, with a theoretical limit of +/−30 degrees. However, these waveguides miss out on the +/−30 to 100 degree ranges that the human eye is capable of seeing, and any light introduced within the +/−25 degrees, being so close to the central fovea region, can be a visual disturbance to the user.

Therefore, a good light therapy device that maps light onto the retina can encompass a much wider field of view compared with conventional augmented reality waveguides. The wide field of view can be analogous to the span of light that a person gets by standing on top of a mountain underneath a blue sky, which is in theory the ideal light therapy condition and span the entire human retina field of view, approximately ~220 degree horizontally and ~135 degree vertically. In addition, a good light therapy device has the ability to project light onto the user's retina in a way that does not interfere with a person's task and color vision. Furthermore, the light therapy device has the ability to be discreet, in that it is not distinguishable to an external observer that the user is wearing anything other than a regular pair of glasses. With the aforementioned design intentions, discloses various methods are disclosed herein to achieve precise, wide field of view light projection to the user's eyes for therapeutic purposes.

Methods, systems, and devices related to light therapy using ophthalmic devices are disclosed. An optical device, with at least one light source, and a system of light guiding mechanisms, enable precise location control of light projection onto a wide field of view on the retina while avoiding the central task vision areas of the retina, while also having the ability to maintain discreteness to an observer. The optical device can use a light source that selectively projects light to the inside of an ophthalmic lens, which has off-axis mirrors that direct light into the user's retina. The optical device can leverage total internal reflection to route light through the ophthalmic lens, and use coupling elements, including partial mirrors, holographic films, or surface reliefs to direct the light toward the user's retina. The optical device can also use a system of mirrors that work together with the light source to direct light via oblique angles onto the retina. The optical device can include an eye tracking system to enable adjustment of light projection based on gaze direction, where sensors work in tandem with the therapeutic light projections as well as light guiding mechanisms to enable power efficient and discreet eye tracking. Any combination of the aforementioned systems can be implemented in tandem, along with conventional waveguide and projection systems in a single optical device.

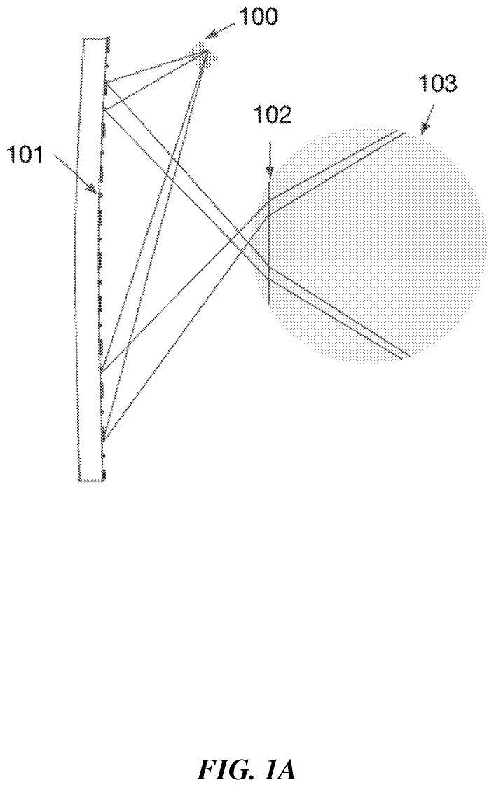

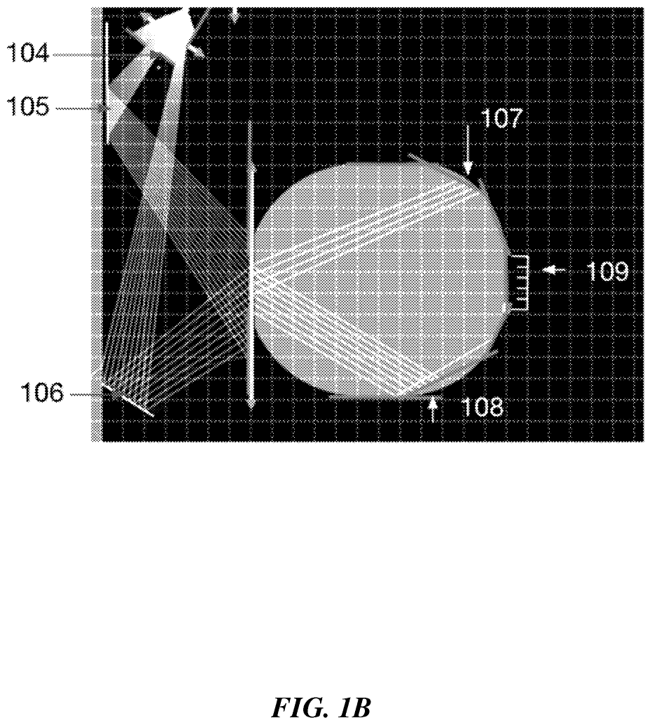

FIG. 1A illustrates an example optical system in accordance with one or more embodiments of the present technology. The optical system presented can be implemented on a pair of spectacles. In this embodiment, a light source 100 and a reflector system 101 work together to direct light towards the human lens 102, such that the light rays land on precise locations on the retina 103.

In some embodiments, the light source 100 comprises a light emitting diode (LED), with or without some imaging optics to shape the beam output. The light source can be turned off or masked at certain locations such that it does not project light onto the selected surface on the lens.

In some embodiments, the light source includes multiple individually controllable sources, such that sections of the light illuminating the back surface of the lens can be turned on or off selectively. One particular method to achieve this is to use a pixelated display as a light source, with a beam shaping optic positioned in front thereof.

In some embodiments, the light source 100 comprises a scanning beam of light that scans across the lens surface. The beam can scan at a high speed and turn on and off at selected time such that certain locations on the lens do not receive light to achieve the effect of masking.

In some embodiments, the reflector system 101 can be implemented as local mirrors mounted on the lens with specific angles to control the reflected ray of light. The mirrors can be on or off axis parabolic mirrors or flat mirrors. The mirrors can be, but not limited to, silvered or semi silvered mirrors, dichroic filmed mirrors, miniature or nano surface mirrors, diffractive optical elements, blaze gratings, holographic films, electrochromic reflectors, or Fresnel reflectors. The mirrors/reflective system can span partial regions on the lens. The mirror(s) on the reflector system 101 can also be adjusted selectively, such that their angle of reflection can be different per each mirror pixel, for example, a digital light processor. In some embodiments, the mirror can be turned on or off selectively. The mirror(s) on the reflector system can have a diffusing element to it, such as to soften/diffuse the light rays entering the eyes.

FIG. 1B illustrates a ray trace diagram in accordance with one or more embodiments of the present technology, showing reflector angles for proper retinal projection. A scanning beam of light 104 is emitted from a light source and is reflected by a reflector system that includes at least reflector element 105 and reflector element 106. In this example, reflector element 105 spans a partial region on the lens and reflector element 106 is mounted at an angle with respect to the lens to control the reflected ray of light such that the light from the reflector system is directed onto the desired locations on the retina (e.g., 107, 108) and avoids the central region 109 to minimize visual disturbance to the user.

An example method to create such a reflector system is to first take a piece of lens and create fine ridge-like patterns on its surface, and then put the lens into a vapor deposition chamber, where reflective surfaces are deposited onto selected locations on the post processed lens. The ridges on the lens can be created by, but not limited to, injection molding, CNC, photolithography, chemical etching, or a combination of processes.

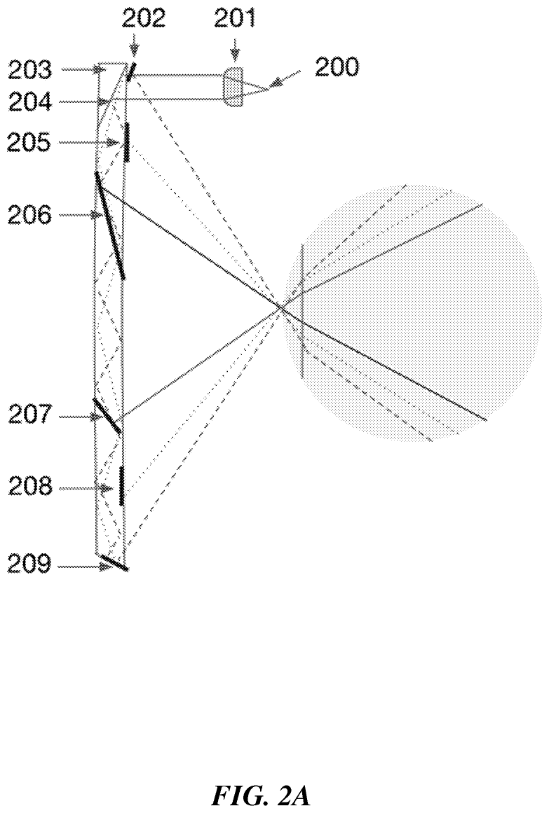

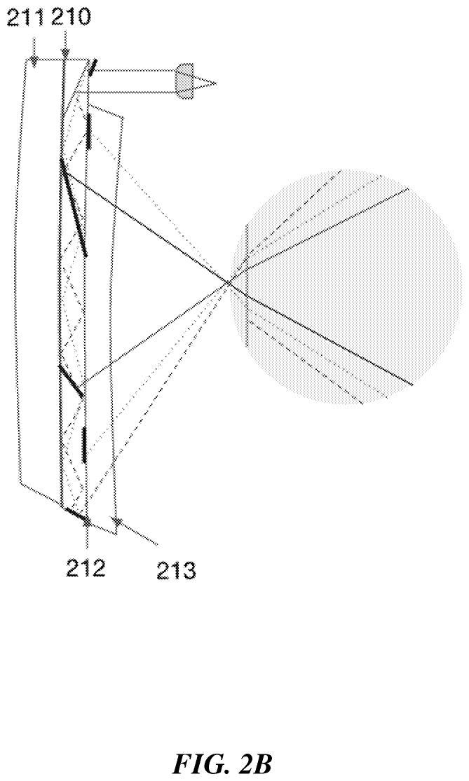

FIG. 2A illustrates an example optical system in accordance with one or more embodiments of the present technology. In this example, light from a source 200 can go through a beam shaper element 201, which is directed or coupled into the lens 203 with a coupler 204 such that the light has total internal reflection and is directed out in a manner to achieve the aforementioned wide angle precision therapy effect. Before reaching the lens, some of the light can be redirected to the eye directly through a mechanism 202 for therapeutic effect. For the light that is going through the lens, some of it can be directed out of the lens via a surface out coupling mechanism 205, 208, which redirect the light from the internal reflection toward the user's eye. The outcoupling mechanism 205 can be, for example, surface gratings, holographic films, or coatings. In some embodiments, some of the light can be redirected by a beam splitter 206, 207, which can be a semi-silvered or dichroic mirror. In some embodiments, some of the light can be redirected by a mirror element 209. What separates this design from conventional waveguide type implementation is that the in-coupling and out-coupling mechanisms are not limited to preserve the image information of the source. By breaking away from that limit, the mechanisms can be designed in a way to provide spatially controlled, wide field of view retina projection focused on light therapy.

In some embodiments, prescription can be enabled through a multilayered lens stack-up design as illustrated in FIG. 2B. As shown in FIG. 2B, a cladding material 210, 212 can be used between the lenses 211, 213. The cladding materials can have a lower refractive index compared with the lens substrate to ensure that total internal reflection is preserved when layers of other substrate materials that support prescription correction are attached.

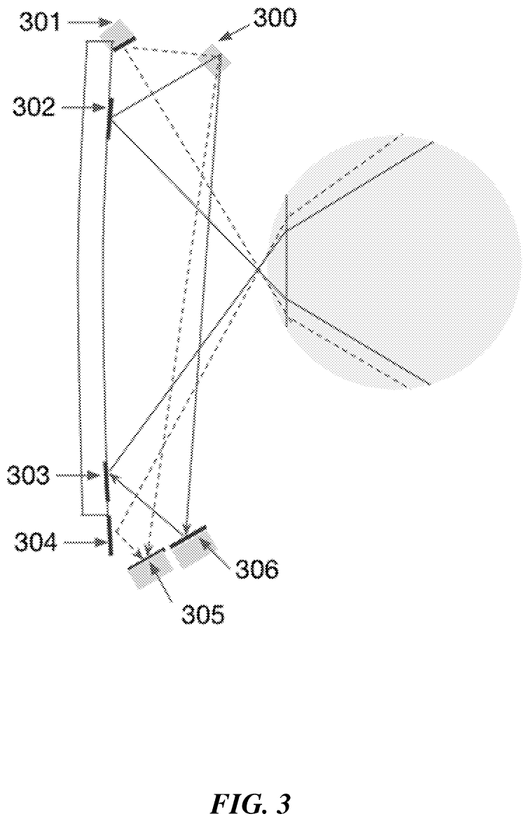

FIG. 3 illustrates another example system in accordance with one or more embodiments of the present technology. In this example, light source(s) 300, along with reflector systems 301, 304, 305, 306 that are mounted onto the frame of the spectacles and reflectors 302, 303 mounted to the lens of the spectacles, direct light into the user's eyes. Reflector systems 301, 304, 305, 306 can also be light sources that either directly project light to the user's eyes, or work with other reflectors in the system that eventually direct light to the retina at oblique angles. In some embodiments, the light source(s) 300 can be a single source, or have the ability to project to multiple locations selectively.

The reflectors 302, 303 on the lens can include a partial dichroic or silvered coating. To enable discreteness, the light source(s) 300 (or 301, 304, 305, 306) can be a narrow band wavelength output source, e.g., lasers, superluminescent diodes, or LEDs, which have another band pass filter window such that only the intended narrowband wavelengths can go through. The reflector 302, 303 can be made in such a way that it achieves near 100% reflection of the intended wavelengths, as to be able to hide the light source to an external observer. For any light that may leak past the coating, another coating or layer of material can be added to hide any leftover light to achieve discreteness.

In some embodiments, an electrochromic coating that is controlled via a circuit can be used to allow the user to adjust its transmission rate from as high as 100% to as low as 0%. The dynamic control of the transmission rate improves the design and user experience, particularly when light blocking is needed to aid with melatonin generation.

In some embodiments, an eye tracking system can be used in tandem with the optical system in order to achieve even better control of light projection to account for eye movements and rotations. Standard eye tracking methods can include camera-based eye tracking, time of flight sensors, and/or ultrasonic sensors. In addition, the light sources used in the design can also serve as part of the eye tracking system to provide illumination and optimize power consumption. In some embodiments, a combination of light sources can be controlled to sweep across the system such as to scan light across the eye at different intervals in time. Light sensors on the frame can use the temporal information from the sweeping light source to calculate the gaze of the eye.

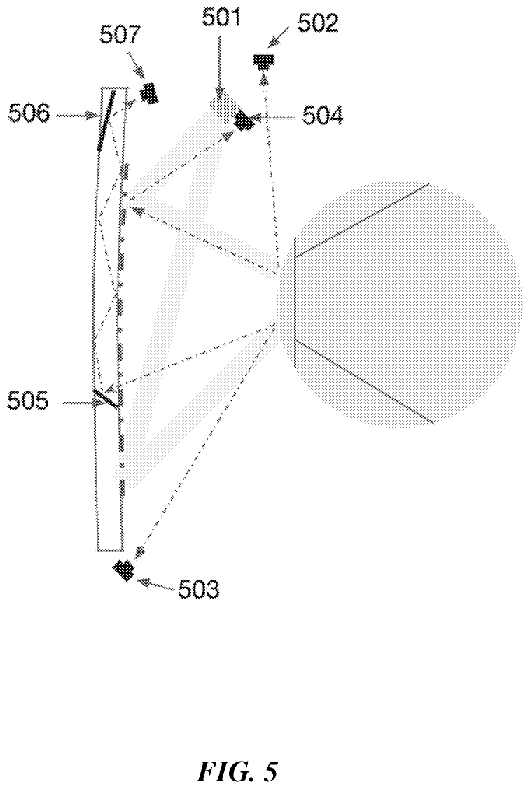

FIG. 5 illustrates an example implementation of an eye tracking system with an optical system in accordance with one or more embodiments of the present technology. This example includes light sources 501 and sensors 502, 503 that are mounted in a way to receive direct light scattered from the cornea. Additional sensor(s) 504 can be configured in a way to receive light that is first reflected from the user's eye to the reflector system on the lens of the device, which then reflects the light to the sensor. In some implementations, a coupler 505 can be used to couple or direct the light reflected from the cornea of the user towards the ophthalmic lens, such that it goes through total internal reflection. An out-coupler 506 can be used to direct the internally reflected light towards a sensor 507.

In some embodiments of the design, all aforementioned methods in this invention can be used in any combination with each other as well as other systems such as waveguide display, or other light therapy systems compatible with the design.

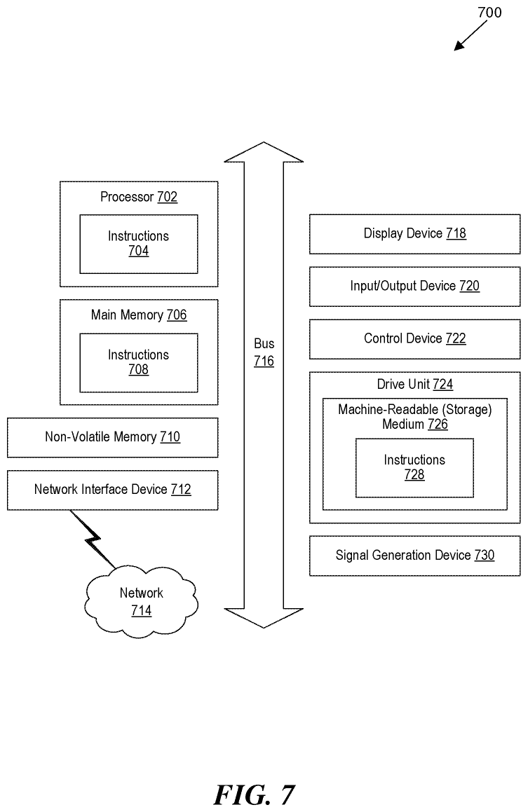

FIG. 7 is a block diagram that illustrates an example of a computing system 700 in which at least some operations described herein (e.g., controller of the optical device to control the coating and/or sensors) can be implemented. As shown, the computing system 700 can include: one or more processors 702, main memory 706, non-volatile memory 710, a network interface device 712, video display device 718, an input/output device 720, a control device 722 (e.g., keyboard and pointing device), a drive unit 724 that includes a storage medium 726, and a signal generation device 730 that are communicatively connected to a bus 716. The bus 716 represents one or more physical buses and/or point-to-point connections that are connected by appropriate bridges, adapters, or controllers. Various common components (e.g., cache memory) are omitted from FIG. 7 for brevity. Instead, the computing system 700 is intended to illustrate a hardware device on which components illustrated or described relative to the examples of the figures and any other components described in this specification can be implemented.

The computing system 700 can take any suitable physical form. For example, the computing system 700 can share a similar architecture as that of a server computer, personal computer (PC), tablet computer, mobile telephone, game console, music player, wearable electronic device, network-connected (“smart”) device (e.g., a television or home assistant device), AR/VR systems (e.g., head-mounted display), or any electronic device capable of executing a set of instructions that specify action(s) to be taken by the computing system 700. In some implementation, the computing system 700 can be an embedded computing system, a system-on-chip (SOC), a single-board computer system (SBC) or a distributed system such as a mesh of computing systems or include one or more cloud components in one or more networks. Where appropriate, one or more computing systems 700 can perform operations in real-time, near real-time, or in batch mode.

The network interface device 712 enables the computing system 700 to mediate data in a network 714 with an entity that is external to the computing system 700 through any communication protocol supported by the computing system 700 and the external entity. Examples of the network interface device 712 include a network adaptor card, a wireless network interface card, a router, an access point, a wireless router, a switch, a multilayer switch, a protocol converter, a gateway, a bridge, bridge router, a hub, a digital media receiver, and/or a repeater, as well as all wireless elements noted herein.

The memory (e.g., main memory 706, non-volatile memory 710, machine-readable medium 726) can be local, remote, or distributed. Although shown as a single medium, the machine-readable medium 726 can include multiple media (e.g., a centralized/distributed database and/or associated caches and servers) that store one or more sets of instructions 728. The machine-readable (storage) medium 726 can include any medium that is capable of storing, encoding, or carrying a set of instructions for execution by the computing system 700. The machine-readable medium 726 can be non-transitory or comprise a non-transitory device. In this context, a non-transitory storage medium can include a device that is tangible, meaning that the device has a concrete physical form, although the device can change its physical state. Thus, for example, non-transitory refers to a device remaining tangible despite this change in state.

Although implementations have been described in the context of fully functioning computing devices, the various examples are capable of being distributed as a program product in a variety of forms. Examples of machine-readable storage media, machine-readable media, or computer-readable media include recordable-type media such as volatile and non-volatile memory devices 710, removable flash memory, hard disk drives, optical disks, and transmission-type media such as digital and analog communication links.

In general, the routines executed to implement examples herein can be implemented as part of an operating system or a specific application, component, program, object, module, or sequence of instructions (collectively referred to as “computer programs”). The computer programs typically comprise one or more instructions (e.g., instructions 704, 708, 728) set at various times in various memory and storage devices in computing device(s). When read and executed by the processor 702, the instruction(s) cause the computing system 700 to perform operations to execute elements involving the various aspects of the disclosure.

Example solutions that implement the disclosed techniques include at least the following:

Solution 1. An optical device, comprising: at least one light source, and a system of light guiding optics configured to direct a light from the at least one light source to a back of a human retina for different therapeutic effects; wherein the light entering the human retina is configured to span a field of view that is approximately up to 220 degrees horizontally and 135 degrees vertically, and wherein the light entering the human retina from the at least one light source is controlled in a manner to avoid a central area that is around +/−10 degrees from a center of the human retina.

2. The optical device of solution 1, wherein the system of light guiding optics is configured to direct the light to an intrinsically photosensitive retinal ganglion cells (ipRGCs) that are present approximately 220 degrees horizontally and 135 degrees vertically of the human retina.

3. The optical device of solution 2, where the optical device is configured to project light that corresponds to a wide field of view of the ipRGCs on the human retina, while selectively avoiding a specific central working vision region of the human retina.

4. The optical device of any of solutions 1 to 3, wherein the different therapeutic effects comprise at least one of: a first therapeutic effect related to circadian rhythm or sleep, or a second circadian rhythm related to depression.

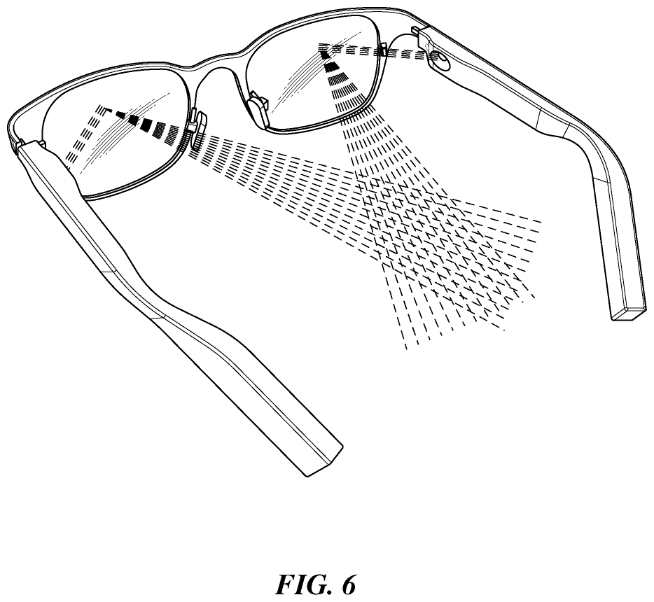

5. The optical device of any of solutions 1 to 4, where the optical device further device comprises: an ophthalmic lens; and a frame comprising a frame front configured to support the ophthalmic lens, the frame comprising two temples configured to allow a user to wear the optical device, wherein the at least one light source is coupled to the frame front or the two temples such as to direct light towards the ophthalmic lens, wherein the system of light guiding optics is mounted onto the frame front or the ophthalmic lens, such as to direct the light from the at least one light source towards to an eye of a user in a manner that allows for high angle of incidence relative to a normal axis of the eye (e.g., as shown in FIG. 6).

6. The optical device of solution 5, wherein the at least one light source comprises an LED, wherein the at least one light source is turned off or masked at certain locations such that it refrains from projecting light onto a selected surface on the ophthalmic lens.

7. The optical device of solution 5 or 6, where the at least one light source comprises multiple individually controllable sources, such that sections of the light illuminating a back surface of the ophthalmic lens are turned on or off selectively.

8. The optical device of any of solutions 5 to 7, wherein the at least one light source is configured to emit a scanning beam of light that scans across a surface of the ophthalmic lens, wherein the scanning beam is configured to scan at a high speed and turn on and off at selected times such that certain locations on the ophthalmic lens are masked from receiving light.

9. The optical device of any of solutions 1 to 8, where the at least one light source comprises a pixelated display with a beam shaping optic in front.

10. The optical device of any of solutions 1 to 9, wherein the system of light guiding optics comprises a system of mirrors that spans partial or an entire region of an ophthalmic lens, wherein the system of mirrors comprises at least one of: silvered or semi silvered mirrors, dichroic filmed mirrors, miniature or nano surface mirrors, diffractive optical elements, blaze gratings, holographic films, Fresnel reflectors, and/or any combination.

11. The optical device of any of solutions 1 to 10, where the system of light guiding optics comprises a diffusing element configured to soften or diffuse light rays entering a user's eyes.

12. The optical device of any of solutions 1 to 11, comprising: an ophthalmic lens; a frame comprising a frame front configured to support the ophthalmic lens, the frame comprising two temples configured to allow a user to wear the optical device (e.g., as shown in FIG. 6); at least one in-coupling mechanism configured to direct light from the at least one light source towards the ophthalmic lens to achieve a total internal reflection of the light; and at least one out-coupling mechanism configured to direct light undergoes the total internal reflection out towards a user's pupil.

13. The optical device of solution 12, wherein the at least one light source comprises an LED, wherein the optical device further comprises a light shaping optic positioned in front of the LED.

14. The optical device of solution 12 or 13, wherein the at least one light source comprises an array of LEDs or a pixelated display, wherein the optical device further comprises a light shaping optic positioned in front of the array of LEDs or the pixelated display.

15. The optical device of any of solutions 12 to 14, wherein part of light from the at least one light source is projected to an eye of the user without undergoing total internal reflection.

16. The optical device of any of solutions 12 to 15, wherein the at least one out-coupling mechanism comprises at least one of surface gratings, films, coatings, dichroic coatings, holographic films, located on an inner or an outer surface of the ophthalmic lens.

17. The optical device of any of solutions 12 to 16, wherein the at least one out-coupling mechanism comprises mirrors or partially reflective mirrors embedded in the ophthalmic lens and at an oblique angle to either an inner or an outer surface of the ophthalmic lens.

18. The optical device of any of solutions 1 to 17, where the optical device is configured to refrain from preserving imaging information of the at least one light source.

19. The optical device of any of solutions 1 to 18, comprising: an ophthalmic lens; a frame comprising a frame front configured to support the ophthalmic lens, the frame comprising two temples configured to allow a user to wear the optical device; and a system of reflectors mounted or positioned on the ophthalmic lens or the frame, wherein the system of reflectors is configured to direct light from the at least one light source to a user.

20. The optical device of solution 19, where the at least one light source is configured to directly project light to the user, or partially project light to the user while other light goes through the system of reflectors.

21. The optical device of solution 19 or 20, wherein the system of reflectors is configured to directly reflect light from a source towards the human retina, or work with other reflectors in the system to eventually direct light to the human retina.

22. The optical device of any of solutions 19 to 21, wherein the system of reflectors comprises a diffusing element configured to soften the light.

23. The optical device of any of solutions 19 to 22, wherein the system of reflectors is positioned on the frame of the device.

24. The optical device of any of solutions 19 to 23, wherein the system of reflectors is positioned on the ophthalmic lens.

25. The optical device of any of solutions 19 to 24, where the system of reflectors comprises one or more semi silvered mirrors or dichroic mirrors.

26. The optical device of any of solutions 19 to 25, where the system of reflectors is configured to reflect substantially 100% of wavelengths of the light from the at least one light source to prevent observers from seeing presence of the at least one light source.

27. The optical device of any of solutions 19 to 26, where an additional layer of material is added to the ophthalmic lens to prevent light from leaking past the system of reflectors.

28. The optical device of any of solutions 19 to 27, comprising a coupler configured to couple the light reflected from a cornea of the user towards the ophthalmic lens, such that the light goes through total internal reflection.

29. The optical device of any of solutions 1 to 28, where the at least one light source comprises a narrow band wavelength output source that outputs wavelengths in selected ranges, the narrow band wavelength output source comprising at least one of a laser, a superluminescent diode, or a LED.

30. The optical device of any of solutions 1 to 29, comprising an eye tracking system that comprises one or more sensors configured to work in tandem with light or reflected light to track a gaze direction of an eye of a user.

31. The optical device of solution 30, where the eye tracking system comprises camera-based eye tracking sensors, time of light sensors, or ultrasonic sensors.

32. The optical device of solution 30 or 31, where the at least one light source is turned on and off or configured to emit light that sweeps across the eye at high frequencies as to not be observable by a user, and wherein the one or more sensors are configured to use temporal processing to determine the gaze direction of the eye based on amount of reflected light it receives at that instance in time.

33. The optical device of any of solutions 30 to 32, wherein at least part of the one or more sensors is positioned to directly receive light reflected from a cornea of the eye.

34. The optical device of any of solutions 30 to 33, wherein at least part of the one or more sensors is configured to receive light that is first reflected from the eye of the user, to a system of reflectors configured to reflect the light to the at least part of the one or more sensors.

35. The optical device of solutions 1 to 34, where comprising multiple prescription lens layers, and wherein a cladding material with a lower refractive index sandwiched between the multiple prescription lens layers to ensure total internal reflection is kept even with multiple prescription lens layers.

Any combination of the solutions above can be implemented in tandem, along with any other combinations of conventional waveguides or projection systems, phase arrays, mirror based laser scanning, electro-optic modulators, risley prisms in a single optical device.

Various operations disclosed herein can be implemented using a processor/controller is configured to include, or be couple to, a memory that stores processor executable code that causes the processor/controller carry out various computations and processing of information. The processor/controller can further generate and transmit/receive suitable information to/from the various system components, as well as suitable input/output (IO) capabilities (e.g., wired or wireless) to transmit and receive commands and/or data.

Various information and data processing operations described herein may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Therefore, the computer-readable media that is described in the present application comprises non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1-35. (canceled)

36. An optical device, comprising:
at least one light source; and
a system of light guiding optics configured to direct light from the at least one light source to a back of a human retina for different therapeutic effects,
wherein the light entering the human retina is configured to span a field of view that is approximately up to 220 degrees horizontally and 135 degrees vertically, and
wherein the light entering the human retina from the at least one light source is controlled in a manner to avoid a central area that is around +/−10 degrees from a center of the human retina.

37. The optical device of claim 36, wherein the system of light guiding optics is configured to direct the light across a wide field of view of the human retina to intrinsically photosensitive retinal ganglion cells (ipRGCs), the wide field of view being approximately 220 degrees horizontally and 135 degrees vertically, while selectively avoiding a central working vision region of the human retina.

38. The optical device of claim 36, wherein the different therapeutic effects comprise at least one of: a first therapeutic effect related to circadian rhythm or sleep, or a second therapeutic effect related to depression.

39. The optical device of claim 36, wherein the optical device further comprises:
an ophthalmic lens; and
a frame comprising a frame front configured to support the ophthalmic lens, the frame comprising two temples configured to allow a user to wear the optical device,
wherein the at least one light source is coupled to the frame front or the two temples such as to direct light towards the ophthalmic lens, and
wherein the system of light guiding optics is mounted onto the frame front or the ophthalmic lens such as to direct the light from the at least one light source toward an eye of a user in a manner that allows for a high angle of incidence relative to a normal axis of the eye.

40. The optical device of claim 39, wherein the at least one light source comprises one or more individually controllable LEDs configured to be selectively turned off or masked at certain locations such that light is refrained from being projected onto selected surfaces of the ophthalmic lens.

41. The optical device of claim 39, wherein the at least one light source is configured to emit a scanning beam of light that scans across a surface of the ophthalmic lens, wherein the scanning beam is configured to scan at a high speed and turn on and off at selected times such that certain locations on the ophthalmic lens are masked from receiving light.

42. The optical device of claim 36, wherein the system of light guiding optics comprises a system of mirrors that spans a partial or an entire region of an ophthalmic lens, wherein the system of mirrors comprises at least one of: silvered or semi-silvered mirrors, dichroic filmed mirrors, miniature or nano-surface mirrors, diffractive optical elements, blaze gratings, holographic films, Fresnel reflectors, and/or any combination thereof.

43. The optical device of claim 36, comprising:
an ophthalmic lens;
a frame comprising a frame front configured to support the ophthalmic lens, the frame comprising two temples configured to allow a user to wear the optical device;
at least one in-coupling mechanism configured to direct light from the at least one light source towards the ophthalmic lens to achieve total internal reflection of the light; and
at least one out-coupling mechanism configured to direct the light undergoing total internal reflection out towards a user's pupil.

44. The optical device of claim 43, wherein the at least one light source comprises one or more LEDs or a plurality of individually addressable light sources, and wherein the optical device further comprises a light shaping optic positioned in front of the at least one light source.

45. The optical device of claim 43, wherein the at least one out-coupling mechanism comprises one or more surface gratings, films, coatings, dichroic coatings, holographic films, mirrors, or partially reflective mirrors located on, in, or embedded within the ophthalmic lens, at least some of which are oriented at an oblique angle relative to an inner or outer surface of the ophthalmic lens to direct light toward the user's pupil.

46. The optical device of claim 36, wherein the optical device is configured to refrain from preserving imaging information of the at least one light source.

47. The optical device of claim 36, comprising:
an ophthalmic lens;
a frame comprising a frame front configured to support the ophthalmic lens, the frame comprising two temples configured to allow a user to wear the optical device; and
a system of reflectors mounted or positioned on the ophthalmic lens or the frame, wherein the system of reflectors is configured to direct light from the at least one light source to a user.

48. The optical device of claim 47, wherein the at least one light source is configured to directly project light to the user, or to project at least part of the light to the user while another part of the light is directed through the system of reflectors, and wherein the system of reflectors is configured to directly reflect light from the at least one light source toward the human retina or to cooperate with one or more other reflectors to direct the light toward the human retina, the system of reflectors being positioned on the frame of the device, on the ophthalmic lens, or both.

49. The optical device of claim 47, wherein the system of reflectors comprises one or more semi-silvered mirrors or dichroic mirrors.

50. The optical device of claim 47, wherein the system of reflectors is configured to reflect substantially 100% of wavelengths of the light from the at least one light source to prevent observers from seeing the presence of the at least one light source.

51. The optical device of claim 47, wherein an additional layer of material is added to the ophthalmic lens to prevent light from leaking past the system of reflectors.

52. The optical device of claim 36, wherein the at least one light source comprises a narrow band wavelength output source that outputs wavelengths in selected ranges, the narrow band wavelength output source comprising at least one of a laser, a superluminescent diode, or an LED.

53. The optical device of claim 36, comprising an eye tracking system comprising one or more sensors configured to work in tandem with light or reflected light to track a gaze direction of an eye of a user.

54. The optical device of claim 53, wherein the one or more sensors comprise camera-based eye tracking sensors, time-of-flight sensors, or ultrasonic sensors, and wherein the at least one light source is configured to emit light in a temporally modulated or sweeping manner such that the one or more sensors determine gaze direction based on reflected light received either directly from a cornea of the eye or indirectly via a system of reflectors.

55. The optical device of claim 36, comprising multiple prescription lens layers, and wherein a cladding material with a lower refractive index is sandwiched between the multiple prescription lens layers to ensure that total internal reflection is maintained even with the multiple prescription lens layers.
